(19) United States
(12) Patent Application Publication
(10) Pub. No.: US 2015/0141376 A1
(43) Pub. Date: May 21, 2015

(54) Pharmaceutical compositions of anti-viral compounds and process for preparation thereof

(71) Applicants: Chandrashekhar Kandi, Hyderabad (IN); Nagaprasad Vishnubhotla, Hyderabad (IN); Asif Anwar, Hyderabad (IN); Krishna Kumar Chegonda, Hyderabad (IN); Sivakumaran Meenakshisunderam, Hyderabad (IN)

(72) Inventors: Chandrashekhar Kandi, Hyderabad (IN); Nagaprasad Vishnubhotla, Hyderabad (IN); Asif Anwar, Hyderabad (IN); Krishna Kumar Chegonda, Hyderabad (IN); Sivakumaran Meenakshisunderam, Hyderabad (IN)

(21) Appl. No.: 14/544,041

(22) Filed: Nov. 18, 2014

(30) Foreign Application Priority Data
Nov. 18, 2013 (IN) IN 5288/CHE/2013

(51) Int. Cl. A61K 9/20; A61K 31/506; A61K 31/675; A61K 31/536

(52) U.S. Cl. CPC A61K 9/2077; A61K 31/536; A61K 31/506; A61K 31/675; A61K 9/2018; A61K 9/2095; A61K 9/2027; A61K 9/2054; A61K 9/2013
USPC 514/81

(57) ABSTRACT

Pharmaceutical compositions of anti-viral compounds, process for preparation and method of using the same are provided. Particularly, the present invention relates to chemically stable pharmaceutical compositions of efavirenz, emtricitabine and tenofovir disoproxil fumarate with optionally one or more pharmaceutically acceptable excipients, process for preparation and method for the treatment or prevention of the symptoms or effects of an HIV infection in an infected patient.

### CROSS REFERENCE TO RELATED APPLICATIONS

This application claims priority from an Indian Patent Application IN 5288/CHE/2013 filed on Nov. 18, 2013.

### FIELD OF THE INVENTION

The present invention relates to pharmaceutical compositions of anti-viral compounds, process for preparation and method of using the same. Particularly, the present invention relates to chemical stable pharmaceutical compositions of efavirenz, emtricitabine and tenofovir disoproxil fumarate, process for preparation and method for the treatment or prevention of the symptoms or effects of an HIV infection in an infected patient.

### BACKGROUND OF THE INVENTION

The management of HIV/AIDS normally includes the use of multiple antiretroviral drugs in an attempt to control HIV infection. There are several classes of drugs, which are usually used in combination, to treat HIV infection. Use of these drugs in combination is generally termed ARTs or Anti-Retroviral Therapy. Anti-retroviral (ARV) drugs are broadly classified by the phase of the retrovirus life-cycle that the drug inhibits. Typical combinations include 2 NRTIs (Nucleoside Reverse Transcriptase Inhibitors)+1 PI (Protease Inhibitor) or 2 NRTIs+1 NNRTI (Non-Nucleoside Reverse Transcriptase Inhibitor).

Antiretroviral combination therapy defends against resistance by suppressing HIV replication as much as possible. Combinations of antiretrovirals create multiple obstacles to HIV replication to keep the number of offspring low and reduce the possibility of a superior mutation. In recent years, drug companies have worked together to combine these complex regimens into simpler formulas. For instance, two pills containing two or three medications each can be taken twice daily. This greatly increases the ease with which they can be taken, which in turn increases adherence, and thus their effectiveness over the long-term. Lack of adherence is a cause of resistance development in medication-experienced patients. Patients who maintain proper therapy can stay on one regimen without developing resistance. This greatly increases life expectancy and leaves more drugs available to the individual should the need arise.

Efavirenz is chemically described as (S)-6-chloro-4-(cyclopropylethynyl)-1,4-dihydro-4-(trifluoromethyl)-2H-3,1-benzoxazin-2-one. Its structural formula is as follows:

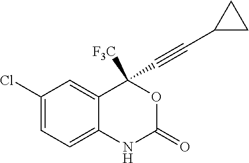 [SMILES: O=C1Nc2ccc(Cl)cc2[C@@](C#CC2CC2)(C(F)(F)F)O1]

Efavirenz is marketed as film-coated tablets for oral administration containing 600 mg of efavirenz and also as capsules in strengths of 50 mg and 200 mg of efavirenz under the brand name SUSTIVA®.

Emtricitabine is chemically described as 5-fluoro-1-(2R,5S)-[2-(hydroxy-methyl)-1,3-oxathiolan-5-yl]cytosine. Its structural formula is as follows:

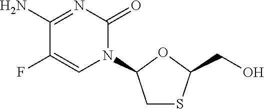 [SMILES: Nc1nc(=O)n([C@@H]2CS[C@H](CO)O2)cc1F]

Emtricitabine is marketed as capsules for oral administration which contains 200 mg of emtricitabine and oral solution for oral administration wherein one 1 mL of emtricitabine oral solution contains 10 mg of emtricitabine in an aqueous solution. Emtricitabine is marketed under the brand name EMTRIVA®.

Tenofovir disoproxil fumarate (a prodrug of tenofovir) which is a fumaric acid salt of bis-isopropoxy carbonyl oxymethyl ester derivative of tenofovir is chemically described as 9-[(R)-2-[[bis[[(isopropoxycarbonyl)oxy]methoxy]phospho-nyl]methoxy]propyl]adenine fumarate (1:1). Its structural formula is as follows:

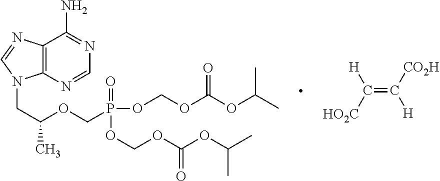 [SMILES: CC(C)OC(=O)OCOP(=O)(CO[C@H](C)Cn1cnc2c(N)ncnc21)OCOC(=O)OC(C)C ; O=C(O)/C=C/C(=O)O]

Tenofovir disoproxil fumarate is marketed as oral tablets in strengths of 150, 200, 250, and 300 mg of tenofovir disoproxil fumarate and also as oral powder which appears as white, taste-masked, coated granules containing 40 mg of tenofovir disoproxil fumarate per gram of oral powder. Tenofovir is marketed under the brand name VIREAD®.

Combination of emtricitabine and tenofovir disoproxil fumarate is marketed under the brand name TRUVADA®. Truvada is marketed as tablets for oral administration. Each film-coated tablet contains 200 mg of emtricitabine and 300 mg of tenofovir disoproxil fumarate.

Combination of efavirenz, emtricitabine and tenofovir disoproxil fumarate is marketed under the brand name ATRIPLA® available only in the form of tablets. ATRIPLA is marketed as tablets for oral administration. Each tablet contains 600 mg of efavirenz, 200 mg of emtricitabine, and 300 mg of tenofovir disoproxil fumarate (which is equivalent to 245 mg of tenofovir disoproxil) as active ingredients. Bristol Myers Squibb originally conceived the concept behind Atripla and along with Gilead jointly developed this triple combination. U.S. Pat. No. 5,519,021 discloses efavirenz. U.S. Pat. Nos. 6,639,071 and 6,939,964 discloses crystalline Form I, Form II and Form III of efavirenz. U.S. Pat. No. 5,814,639 discloses emtricitabine. U.S. Pat. Nos. 5,922,695 and 5,977,089 discloses tenofovir disoproxil. U.S. Pat. No. 5,935,946 discloses tenofovir disoproxil fumarate.

U.S. Patent Application Publication No. 2004/0224917 discloses combination of tenofovir and emtricitabine for treatment of I-IIV infections. This patent publication further discloses a combination of tenofovir, emtricitabine and efavirenz compositions in fixed dose combination or in the form of patient pack. U.S. Patent Application Publication No. 2007/0077295 A1 discloses dry granulated compositions of emtricitabine and tenofovir disoproxil fumarate. The said application further discloses triple combination of emtricitabine, tenofovir disoproxil fumarate and efavirenz by dry and wet granulation.

U.S. Patent Application Publication No. 2007/0099902 A1 discloses composition comprising tenofovir disoproxil fumarate and a surfactant whereby the surfactant is in a stabilizing configuration with tenofovir disoproxil fumarate. The said application further exemplifies bilayer tablet compositions of efavirenz, emtricitabine and tenofovir disoproxil fumarate.

As per the disclosure of '902 patent application publication, use of surfactant is inevitable in the composition comprising combination of efavirenz, emtricitabine and tenofovir disoproxil fumarate. Absence of surfactant in the composition in a tablet failed to achieve bioequivalence with respect to efavirenz in human clinical trials. Further efavirenz formulation was found to be unexpectedly incompatible with tenofovir disoproxil fumarate due to presence of surfactant in the efavirenz formulation. To overcome the incompatibility issues of tenofovir with the surfactant and at the same time fulfil the requirement for the need of surfactant in efavirenz formulation, the inventors of '902 patent application publication developed the components of the dosage form conveniently organized in multiple layers, preferably bilayer tablet dosage form. However, the process disclosed in '902 patent is cumbersome, requires multiple processing steps, special tablet compression machine, and needs increased man power and processing time, which results to an expensive product.

International Publication No. WO 2008/096369 A2 discloses monolithic tablet formulation comprising a) a nucleotide analog reverse transcriptase inhibitor (NtRTI); b) a non-nucleoside reverse transcriptase inhibitors (NNRTI); c) a nucleoside analog reverse transcriptase inhibitors (NRTI) and d) one or more pharmaceutically acceptable carriers or excipients. This patent publication further discloses process for preparation of monolithic tablet formulation comprising efavirenz, emtricitabine and tenofovir disoproxil fumarate; wherein the efavirenz granules are prepared individually; and tenofovir and emtricitabine are mixed together to prepare granules. These two granule compositions are mixed and the mixture is then compressed and coated. The said patent publication discloses compositions wherein efavirenz component is either wet or dry granulated and emtricitabine and tenofovir components are mixed together and then compacted. The said process has the main disadvantage in the fact that dry granulation in the form of slugging and compacts results in more dust than wet granulation, thus increasing the chances of contamination. Based on available prior arts, it can be inferred that there is still a need to follow a cost effective and alternative process which saves considerable amount of time, is efficient, economical and decreases the chances of contamination. Further, economical HIV therapy formulations would tend to reduce the final prices for AIDS patients across the world, especially in third world and developing countries. Thus inventors of the present invention have developed pharmaceutical compositions of efavirenz, emtricitabine and tenofovir disoproxil fumarate using alternative process which results economical dosage form and decreases the chances of contamination.

### SUMMARY OF THE INVENTION

An aspect of the present invention relates to pharmaceutical compositions comprising therapeutically effective amount of efavirenz, emtricitabine and tenofovir or pharmaceutically acceptable salt thereof as active ingredients, optionally comprising one or more pharmaceutically acceptable excipient(s).

Another aspect of the present invention relates to pharmaceutical compositions comprising therapeutically effective amount of efavirenz, emtricitabine and tenofovir or pharmaceutically acceptable salt thereof as active ingredients, optionally comprising one or more pharmaceutically acceptable excipient(s) wherein the said composition is in the form of a single layer tablet.

Yet another aspect of the present invention relates to pharmaceutical compositions comprising therapeutically effective amount of efavirenz, emtricitabine and tenofovir or pharmaceutically acceptable salt thereof as active ingredients, optionally comprising one or more pharmaceutically acceptable excipient(s) as a single layer tablet wherein Tenofovir disoproxil fumarate, Efavirenz and Emtricitabine are each present in an amount from about 100 mg to about 600 mg.

Another aspect of the present invention relates to pharmaceutical compositions comprising therapeutically effective amount of efavirenz, emtricitabine and tenofovir or pharmaceutically acceptable salt thereof optionally comprising one or more pharmaceutically acceptable excipient(s) as a single layer tablet wherein the single layer tablet comprises about 300 mg of Tenofovir disoproxil fumarate, about 600 mg of Efavirenz and about 200 mg of Emtricitabine.

Another aspect of the present invention relates to a process for the preparation of pharmaceutical compositions, wherein the process comprises of the following steps:

i) preparing the efavirenz fraction separately, optionally with one or more pharmaceutically acceptable excipient(s),
ii) preparing the emtricitabine fraction separately, optionally with one or more pharmaceutically acceptable excipient(s),
iii) preparing the tenofovir fraction separately, optionally with one or more pharmaceutically acceptable excipient(s), and
iv) formulating the material of steps (i), (ii) and (iii) into a single layer tablet.

Another aspect of the present invention relates to a process for the preparation of pharmaceutical compositions, wherein the process comprises of the following steps:

i) preparing the efavirenz fraction separately, optionally with one or more pharmaceutically acceptable excipient(s),
ii) treating emtricitabine and tenofovir fractions together, optionally with one or more pharmaceutically acceptable excipient(s), and
iii) formulating the material of steps (i) and (ii) into a single layer tablet.

An aspect of the present invention relates to method of using such compositions for treatment of patients infected with HIV that provides enhanced therapeutic safety and efficacy, impart lower resistance, and results in higher patient compliance.

### DETAILED DESCRIPTION OF THE INVENTION

The present invention relates to pharmaceutical compositions comprising therapeutically effective amount of efavirenz, emtricitabine and tenofovir or pharmaceutically acceptable salt thereof as active ingredients and optionally comprising one or more pharmaceutically acceptable excipient(s).

The term “composition” or “pharmaceutical composition” or “dosage form” as used herein synonymously include solid dosage forms such as granules, multiunit particulate systems (MUPS), pellets, spheres, tablets, capsules, mini-tablets, beads, particles and the like; and liquid dosage forms such as solutions, suspensions, emulsions, colloids and the like, meant for oral administration.

The term “therapeutically effective amount” is defined to mean the amount or quantity of the active drug (e.g. efavirenz, emtricitabine, tenofovir or a combination thereof), which is sufficient to elicit an appreciable biological response when administered to the patient. The term “active ingredient(s)” refers to include efavirenz, emtricitabine and tenofovir throughout the specification unless otherwise stated in the specification.

In accordance with the present invention, the term “efavirenz” includes efavirenz in the form of free base or its pharmaceutically acceptable salt, amorphous, crystalline or any isomer or derivative, hydrate or solvate, prodrug or combinations thereof. Preferably efavirenz is in the form of free base. In accordance with the present invention, the term “emtricitabine” includes emtricitabine in the form of free base or its pharmaceutically acceptable salt, amorphous, crystalline or any isomer or derivative, hydrate or solvate, prodrug or combinations thereof. Preferably emtricitabine is in the form of free base. In accordance with the present invention, the term “tenofovir” includes tenofovir in the form of free base or its pharmaceutically acceptable salt, amorphous, crystalline or any isomer or derivative, hydrate or solvate, prodrug or combinations thereof. Preferably tenofovir is in the form of tenofovir disoproxil fumarate. The phrase “substantially pure polymorphic form of efavirenz or emtricitabine or tenofovir”, unless otherwise specified is to be understood as a substance free of other polymorphic and/or pseudopolymorphic forms at amounts detectable with typical analytical methods such as X-ray powder diffraction and/or solid state infrared absorption, i.e. containing less than 10% of other polymorphic and/or pseudopolymorphic forms.

The term “excipient” means a pharmacologically inactive component such as a diluent, disintegrant, carrier, or the like. The excipients that are useful in preparing a pharmaceutical composition are generally safe, non-toxic and are acceptable for veterinary as well as human pharmaceutical use. Reference to an excipient includes both one and more than one such excipient.

As used in this specification, the singular forms “a”, “an”, and “the” include plural references unless the context clearly dictates otherwise. Thus for example, a reference to “a process” includes one or more process, and/or steps of the type described herein and/or which will become apparent to those persons skilled in the art upon reading this disclosure and so forth.

In an embodiment, the present invention relates to pharmaceutical compositions comprising therapeutically effective amount of efavirenz, emtricitabine and tenofovir or pharmaceutically acceptable salt thereof as active ingredients, optionally comprising one or more pharmaceutically acceptable excipient(s).

In another embodiment, the present invention relates to pharmaceutical compositions comprising therapeutically effective amount of efavirenz, emtricitabine and tenofovir or pharmaceutically acceptable salt thereof as active ingredients, optionally comprising one or more pharmaceutically acceptable excipient(s) wherein the said composition is in the form of a single layer tablet.

Yet another embodiment of the present invention relates to pharmaceutical compositions comprising therapeutically effective amount of efavirenz, emtricitabine and tenofovir or pharmaceutically acceptable salt thereof as active ingredients, optionally comprising one or more pharmaceutically acceptable excipient(s) as a single layer tablet wherein Tenofovir disoproxil fumarate, Efavirenz and Emtricitabine are each present in an amount from about 100 mg to about 600 mg.

In another embodiment, the present invention relates to pharmaceutical compositions comprising therapeutically effective amount of efavirenz, emtricitabine and tenofovir or pharmaceutically acceptable salt thereof as active ingredients, optionally comprising one or more pharmaceutically acceptable excipient(s) as a single layer tablet wherein the single layer tablet comprises about 300 mg of Tenofovir disoproxil fumarate, about 600 mg of Efavirenz and about 200 mg of Emtricitabine.

In an embodiment, the composition of the present invention comprises a surfactant. In another embodiment, the ratio of efavirenz fraction to surfactant is about 50:1 to about 10:1. In another embodiment, the tenofovir fraction of the present invention is essentially free of surfactant(s).

In an embodiment, the present invention relates to a process for the preparation of pharmaceutical compositions, wherein the process comprises of the following steps:

i) preparing the efavirenz fraction separately, optionally with one or more pharmaceutically acceptable excipient(s),
ii) preparing the emtricitabine fraction separately, optionally with one or more pharmaceutically acceptable excipient(s),
iii) preparing the tenofovir fraction separately, optionally with one or more pharmaceutically acceptable excipient(s), and
iv) formulating the material of steps (i), (ii) and (iii) into a single layer tablet.

In yet another embodiment, the present invention relates to a process for the preparation of pharmaceutical compositions, wherein the process comprises of the following steps:

i) preparing the efavirenz fraction separately, optionally with one or more pharmaceutically acceptable excipient(s),
ii) preparing the emtricitabine and tenofovir fractions together, optionally with one or more pharmaceutically acceptable excipient(s), and
iii) formulating the material of steps (i) and (ii) into a single layer tablet.

In an embodiment, the present invention relates to a process for the preparation of pharmaceutical compositions, wherein the process comprises of the following steps:

i) treating the efavirenz fraction separately with an aqueous or non-aqueous solvent, optionally with one or more other pharmaceutically acceptable excipient(s),
ii) treating the emtricitabine fraction separately with an aqueous or non-aqueous solvent, optionally with one or more other pharmaceutically acceptable excipient(s),
iii) treating the tenofovir fraction separately with an aqueous or non-aqueous solvent, optionally with one or more other pharmaceutically acceptable excipient(s), and
iv) formulating the material of steps (i), (ii) and (iii) into a single layer tablet.

In another embodiment, the present invention relates to a process for the preparation of pharmaceutical compositions, wherein the process comprises of the following steps:

i) treating the efavirenz fraction separately with an aqueous or non-aqueous solvent, optionally with one or more other pharmaceutically acceptable excipient(s),
ii) treating the emtricitabine fraction with an aqueous or non-aqueous solvent and tenofovir fractions together, optionally with one or more other pharmaceutically acceptable excipient(s), and
iii) formulating the material of steps (i) and (ii) into a single layer tablet.

In another embodiment, the present invention relates to a process for the preparation of pharmaceutical compositions, wherein the process comprises of the following steps:

i) treating efavirenz fraction separately with an aqueous solvent, optionally with one or more other pharmaceutically acceptable excipient(s),
ii) treating emtricitabine fraction separately with non-aqueous solvent, optionally with one or more other pharmaceutically acceptable excipient(s),
iii) treating tenofovir fraction separately with non-aqueous solvent, optionally with one or more other pharmaceutically acceptable excipient(s), and
iv) formulating the material of steps (i), (ii) and (iii) into a single layer tablet.

In an embodiment, the present invention relates to a process for the preparation of pharmaceutical compositions, wherein the process comprises of the following steps:

i) treating efavirenz fraction separately with an aqueous solvent, optionally with one or more other pharmaceutically acceptable excipient(s),
ii) treating emtricitabine fraction with aqueous solvent and tenofovir fraction with a non-aqueous solvent together, optionally with one or more other pharmaceutically acceptable excipient(s), and
iii) formulating into a single layer tablet.

In another embodiment, the present invention relates to a process for the preparation of pharmaceutical compositions, wherein the process comprises of the following steps:

i) treating efavirenz fraction separately with an aqueous solvent, optionally with one or more other pharmaceutically acceptable excipient(s),
ii) treating the emtricitabine fraction with non-aqueous solvent and tenofovir fractions together, optionally with one or more other pharmaceutically acceptable excipient(s), and
iii) formulating into a single layer tablet.

In an embodiment, the present invention also provides single layer tablet dosage form comprising about 0.1% w/w to about 99% w/w of efavirenz, emtricitabine and tenofovir as active ingredient(s) and optionally with one or more pharmaceutically acceptable excipients from about 0.1% to about 99% based on total weight of the composition. In an embodiment, the pharmaceutical composition of the present invention can be prepared by either direct compression, dry compression (slugging), or by granulation, preferably wet granulation. The wet granulation technique is either aqueous or non-aqueous. In an embodiment, the equipment used for the granulation are selected from but not limited to rapid mixer granulators, fluidized bed granulators or the like. It must be appreciated that person skilled in the art would know the possible variations and modifications in the type of equipment used in the manufacturing process and are in the scope of the instant invention.

In an embodiment, the tablet compositions of the present invention are film coated. A film forming agent may provide smooth film-forming coating suspensions and enhance the rheological mechanical strength properties of film coating gel matrices. Film forming agents include, for example, polyvinyl pyrrolidone, natural gums, starches, and cellulosic polymers. A cellulosic polymer may include a molecule comprising at least one cellulose polymer or derivative modified with small amounts of propylene glycol ether groups attached to the cellulose anhydroglucose chain affording binding properties that enhance the reinforcing film properties of film applications. Examples of cellulosic polymers include, but are not limited to, hydroxypropyl methyl cellulose (“HPMC”), carboxymethyl cellulose (“CMC”) or salts thereof, hydroxypropyl cellulose (“HPC”), methylcellulose (“MC”), hydroxyethyl cellulose (“HEC”), and the like. In addition, cellulosic polymers may be characterized as ionic or non-ionic. Ionic cellulosic polymers include, for example, sodium CMC. Non-ionic cellulosic polymers include, for example, HPMC, HPC, HEC, and MC. A variety of commercially available cellulosic polymers exists and may include, for example, Spectracel® HPMC compositions (available from Sensient Technologies).

Useful pharmaceutical excipients according to the present invention include diluents, binders, disintegrants, surfactants, glidant, lubricants, glidants/antiadherants; chelating agents; vehicles; bulking agents; stabilizers; preservatives and a combination thereof. It might be appreciated that the selection of pharmaceutical excipients useful in the compositions of the present invention are selected from but not limited to a group of excipients generally known to persons skilled in the art.

Exemplary “diluents” include, but are not limited to, microcrystalline cellulose, lactose, sugar, starches, modified starches, pregelatinized starch, talc, kaolin, sucrose, mannitol, sorbitol, dextrates, dextrin, maltodextrin, dextrose, mannitol, sorbitol, xylitol, lactitol, calcium carbonate, calcium sulfate, dibasic calcium phosphate, tribasic calcium phosphate, magnesium carbonate, magnesium oxide and the like used either alone or in combinations thereof. Exemplary “binders” include, but are not limited to, hydroxypropyl cellulose, hydroxypropyl methylcellulose, low-substituted hydroxypropyl cellulose, povidone, starches such as corn starch, potato starch, modified starches, sugars, guar gum, pectin, wax binders, methylcellulose, carboxymethylcellulose, hydroxyethyl cellulose, copolyvidone, carboxymethylcellulose sodium, ethyl cellulose, gelatin, liquid glucose and pregelatinized starch and the like used either alone or in combinations thereof.

Exemplary “disintegrants” include, but are not limited to, croscarmellose sodium, crospovidone, sodium starch glycolate, polacrilin potassium, microcrystalline cellulose, pregelatinized maize starch and the like used either alone or in combinations thereof. Exemplary “surfactants” include, but are not limited to, sodium lauryl sulfate, polyethylene glycols, polyethylene glycol fatty acid esters such as PEG monolaurate, PEG dilaurate, PEG distearate, PEG dioleate; polyoxyethylene alkylaryl ethers such as polyoxyethylene lauryl ether, polyoxyethylene acetyl ether, polyoxyethylene stearyl ether; polyoxyethylenesorbitan fatty acid ester such as polysorbate 40, polysorbate 60, polysorbate 80; sorbitan fatty acid mono esters such as sorbitan monolaurate, sorbitan monooleate, sorbitan sesquioleate, sorbitan trioleate, poloxamers, polyoxyethylene castor oil derivates such as polyoxyl castor oil, polyoxyl hydrogenated castor oil and the like used either alone or in combinations thereof.

Exemplary “glidants” include, but are not limited to, colloidal silica, calcium silicate, magnesium silicate, silicon hydrogel, cornstarch, talc, corn starch, DL-leucine and the like used either alone or in combinations thereof. Exemplary “lubricants” include, but are not limited to, magnesium stearate, calcium stearate, sodium stearyl fumarate, zinc stearate, stearic acid, fumaric acid, palmitic acid, talc, carnauba wax, hydrogenated vegetable oils, mineral oil, polyethylene glycols and the like used either alone or in combinations thereof. Exemplary “granulating solvents” used in preparation of the formulations are not limited to, aqueous solvents such as purified water, and non-aqueous solvents such as isopropyl alcohol, dichloromethane, ethanol, acetone, methylene chloride and the like used either alone or in combinations thereof.

The vehicles suitable for use in the present invention can be selected from but not limited to a group comprising dimethyl acetamide, dimethyl formamide and dimethyl sulphoxide, N-methyl pyrrolidone, benzyl benzoate, benzyl alcohol, ethyl oleate, polyoxyethylene glycolated castor oils (commercially available as Cremophor™), polyethylene glycol MW 200 to 6000, propylene glycol, hexylene glycols, butylene glycols and glycol derivatives such as polyethylene glycol 660 hydroxystearate (commercially available as Solutrol® HS 15). In another embodiment of the present invention, the compositions may additionally comprise an antimicrobial preservative such as Benzyl alcohol preferably at a concentration of 2.0% v/v of the composition. In an embodiment of the present invention, the composition may additionally comprise a conventionally known antioxidant such as ascorbyl palmitate, butyl hydroxy anisole, butyl hydroxy toluene, propyl gallate and alpha-tocopherol.

In an embodiment, the tablet compositions of the present invention may be film coated. A film forming agent may provide smooth film-forming coating suspensions and enhance the rheological mechanical strength properties of film coating gel matrices. Film forming agents include, for example, polyvinylpyrrolidone, natural gums, starches, and cellulosic polymers. A cellulosic polymer may include a molecule comprising at least one cellulose polymer or derivative modified with small amounts of propylene glycol ether groups attached to the cellulose anhydroglucose chain affording binding properties that enhance the reinforcing film properties of film applications. Examples of cellulosic polymers include, but are not limited to, hydroxypropyl methyl cellulose (“HPMC”), carboxymethyl cellulose (“CMC”) or salts thereof, hydroxypropyl cellulose (“HPC”), methylcellulose (“MC”), hydroxyethyl cellulose (“HEC”), and the like. In addition, cellulosic polymers may be characterized as ionic or non-ionic. Ionic cellulosic polymers include, for example, sodium CMC. Non-ionic cellulosic polymers include, for example, HPMC, HPC, HEC, and MC. Varieties of commercially available cellulosic polymers exist and may include, for example, Spectracel® HPMC compositions (available from Sensient Technologies). Further, other commercially available coating materials are available marketed under the brand name Opadry® for example Opadry II Gray which contains: lactose monohydrate NF, hypromellose type 2910 USP, titanium dioxide USP, triacetin USP, and iron oxide black JPE; Opadry II Pink which contains: hypromellose type 2910 USP, titanium dioxide USP, lactose monohydrate NF, polyethylene glycol 3350 NF, triacetin USP, and FD&C Red #40; Opadry II Blue which contains: hypromellose type 2910 USP, lactose monohydrate NF, FD&C Blue #1, polyethylene glycol 3350 NF, FD&C Blue #2, titanium dioxide USP, triacetin USP, and D&C Yellow #10; Opadry II Yellow which contains: hypromellose type 2910 USP, lactose monohydrate NF, titanium dioxide USP, iron oxide yellow NF, polyethylene glycol 3350 NF, and triacetin USP; Opadry II Purple which contains: hypromellose type 2910 USP, lactose monohydrate NF, titanium dioxide USP, D&C Red #27, polyethylene glycol 3350 NF, triacetin USP, and FD&C Blue #1 and the like.

The compositions of the present invention can be packed into suitable containers such as bottles, blisters or pouch. Further, the packages may optionally contain a dessicant or an antioxidant or oxygen absorbant or combinations thereof.

In another embodiment, the present invention provides method of using such compositions for treatment of patients infected with HIV that provides enhanced therapeutic safety and efficacy, impart lower resistance, and results in higher patient compliance.

The following examples serve to illustrate the embodiments of the present invention. However, they do not intend to limit the scope of the invention. It is obvious to those skilled in the art to find out the composition for other dosage forms and substitute the equivalent excipients as described in this specification or with the one known to the industry.

### Example-1

Tablet composition comprising efavirenz, emtricitabine and tenofovir disoproxil fumarate:

| S. No | Ingredients | Qty (% w/w) |
|---|---|---|
| | A. Efavirenz fraction: | |
| 1 | Efavirenz | 39.22 |
| 2 | Lactose | 6.21 |
| 3 | Crospovidone | 2.94 |
| 4 | Povidone | 1.31 |
| 5 | Low-substituted hydroxypropyl | 1.31 |
| | cellulose | |
| | Granulation: | |
| 6 | Sodium Lauryl Sulfate | 1.31 |
| 7 | Purified Water^{$} | q.s |
| | B. Emtricitabine + Tenofovir | |
| | disoproxil fumarate fraction: | |
| 8 | Tenofovir disoproxil fumarate | 19.61 |
| 9 | Lactose | 7.84 |
| | Granulation: | |
| 10 | Emtricitabine | 13.07 |
| 11 | Povidone | 2.61 |
| 12 | Isopropyl alcohol^{$} | q.s |
| | C. Extragranular Fraction: | |
| 13 | Crospovidone | 3.59 |
| 14 | Magnesium stearate | 0.98 |
| | Core tablet weight | 100.00 |
| | D. Film coating: | |
| 15 | Opadry White 85F18422 | 3.00 |
| 16 | Purified water^{$} | q.s |

^{$}Lost in processing.

Manufacturing Process:

A. Efavirenz Fraction:

(i) Efavirenz, lactose, crospovidone, povidone and low-substituted hydroxypropyl cellulose were sifted and blended together to form a dry blend,
(ii) Binder solution was prepared by dissolving sodium lauryl sulfate in purified water,
(iii) The blend of step (i) was granulated with binder solution of step (ii), using rapid mixer granulator,
(iv) The granules of step (iii) were dried and milled to get the desired granules of efavirenz.

B. Emtricitabine+Tenofovir Disoproxil Fumarate Fraction:

(i) Tenofovir disoproxil fumarate and lactose were sifted and blended together to form a dry blend,
(ii) Binder solution was prepared by dissolving emtricitabine and povidone in isopropyl alcohol,
(iii) The blend of step (i) was granulated with the with solution of step (ii), using fluid bed processor,
(iv) The granules of step (iii) were dried and milled to get the desired granules of emtricitabine and tenofovir disoproxil fumarate.

C. Extragranular Fraction and Compression:

(i) Efavirenz fraction of step A and emtricitabine and tenofovir disoproxil fumarate fraction of step B were blended together with extragranular crospovidone,
(ii) The blend of step (i) was lubricated with magnesium stearate,
(iii) The lubricated blend of step (ii) was compressed into single layer tablets using suitable compression machine.

D. Film Coating:

Single layer tablets obtained in Step C (iii) were film coated using the dispersion of Opadry II White 85F18422.

### Examples-2 to 3a

Tablet composition comprising efavirenz, emtricitabine and tenofovir disoproxil fumarate

| | | Example 2 | Example 3 | Example 3a |
|---|---|---|---|---|
| | | Qty | Qty | Qty |
| S. No | Ingredients | (% w/w) | (% w/w) | (% w/w) |
| | A. Efavirenz Fraction: | | | |
| 1 | Efavirenz | 39.22 | 39.22 | 39.22 |
| 2 | Mannitol | 6.21 | — | — |
| 3 | Lactose monohydrate | — | 6.21 | 6.21 |
| 4 | Crospovidone | 2.94 | — | — |
| 5 | Sodium starch glycolate | — | 2.94 | 2.94 |
| 6 | Povidone | 1.31 | 1.31 | 1.31 |
| 7 | Low-substituted | 1.31 | 1.31 | 1.31 |
| | hydroxypropyl cellulose | | | |
| | Granulation: | | | |
| 8 | Polyethylene glycol | 1.31 | — | — |
| 9 | Sodium Lauryl Sulfate | — | 1.31 | 1.31 |
| 10 | Purified Water^{$} | q.s | q.s | q.s |
| | B. Emtricitabine Fraction: | | | |
| 11 | Emtricitabine | 13.07 | 13.07 | 13.07 |
| 12 | Lactose | 2.61 | — | — |
| 13 | Microcrystalline cellulose | — | 1.96 | 1.96 |
| 14 | Crospovidone | 1.31 | — | — |
| 15 | Sodium starch glycolate | — | 1.96 | 1.96 |
| | Granulation: | | | |
| 16 | Purified Water^{$} | q.s. | — | q.s. |
| 17 | Isopropyl alcohol^{$} | — | q.s. | — |
| | C. Tenofovir disoproxil | | | |
| | fumarate Fraction: | | | |
| 18 | Tenofovir disoproxil | 19.61 | 19.61 | 19.61 |
| | fumarate | | | |
| 19 | Lactose | 3.92 | 4.57 | 4.57 |
| | Granulation: | | | |
| 20 | Hydroxypropyl methyl | 2.61 | — | — |
| | cellulose (Low | | | |
| | viscosity grade) | | | |
| 21 | Polyethylene glycol 8000 | — | 1.96 | 1.96 |
| 22 | Isopropyl alcohol^{$} | q.s | q.s | — |
| 23 | Dichloromethane^{$} | q.s. | — | — |
| 24 | Purified water^{$} | — | — | q.s. |
| | D. Extragranular Fraction: | | | |
| 25 | Crospovidone | 3.59 | — | — |
| 26 | Sodium starch glycolate | — | 3.59 | 3.59 |
| 27 | Magnesium Stearate | 0.98 | 0.98 | 0.98 |
| | Core tablet weight | 100.00 | 100.00 | 100.00 |

^{$}Lost in processing.

Manufacturing Process:

A. Efavirenz Fraction:

(i) Efavirenz, mannitol, crospovidone, povidone and low-substituted hydroxypropyl cellulose were sifted and blended together to form a dry blend,
(ii) Binder solution was prepared by dissolving polyethylene glycol in purified water,
(iii) The blend of step (i) was granulated with solution of step (ii), using rapid mixer granulator,
(iv) The granules of step (iii) were dried and milled to get the desired granules of efavirenz.

B. Emtricitabine Fraction:

(i) Emtricitabine, lactose, crospovidone were sifted and blended together to form a dry blend,
(ii) The blend of step (i) was granulated with purified water, using rapid mixer granulator,
(iii) The granules of step (ii) were dried and milled to get the desired granules of emtricitabine.

C. Tenofovir Disoproxil Fumarate Fraction:

(i) Tenofovir disoproxil fumarate and lactose were sifted and blended together to form a dry blend,
(ii) Hydroxypropyl methylcellulose solution was prepared in a mixture of isopropyl alcohol and dichloromethane,
(iii) The blend of step (i) was granulated with solution of step (ii), using fluid bed processor,
(iv) The granules of step (iii) were dried and milled to get the desired granules of tenofovir disoproxil fumarate.

D. Extragranular Fraction and Compression:

(i) Efavirenz fraction of step A, emtricitabine fraction of step B and tenofovir disoproxil fumarate fraction of step C were blended with extragranular crospovidone,
(ii) The blend of step (i) was lubricated using magnesium stearate,
(iii) The lubricated blend of step (ii) was compressed using suitable compression machine.

### Example-3 & 3a

### Manufacturing Process Similar to Example-2

### Example-4

Tablet composition comprising efavirenz, emtricitabine and tenofovir disoproxil fumarate

| S. No | Ingredients | Qty (% w/w) |
|---|---|---|
| | A. Efavirenz Fraction: | |
| 1 | Efavirenz | 39.22 |
| 2 | Microcrystalline cellulose | 6.21 |
| 3 | Croscarmellose sodium | 2.94 |
| 4 | Hydroxypropyl cellulose | 1.31 |
| 5 | Low-substituted hydroxypropyl | 1.31 |
| | cellulose | |
| | Granulation: | |
| 6 | Sodium Lauryl Sulfate | 1.31 |
| 7 | Purified Water^{$} | q.s |
| | B. Emtricitabine + Tenofovir | |
| | disoproxil fumarate Fraction: | |
| 8 | Tenofovir disoproxil fumarate | 19.61 |
| 9 | Microcrystalline cellulose | 7.84 |
| | Granulation: | |
| 10 | Emtricitabine | 13.07 |
| 11 | Hydroxypropyl methylcellulose | 2.61 |
| 12 | Isopropyl alcohol^{$} | q.s |
| | C. Extragranular Fraction: | |
| 13 | Croscarmellose sodium | 3.59 |
| 14 | Magnesium Stearate | 0.98 |
| | Core tablet weight | 100.00 |
| | D. Film coating: | |
| 15 | Opadry II White 85F18422 | 3.00 |
| 16 | Purified water^{$} | q.s |

^{$}Lost in processing.

Manufacturing Process:

A. Efavirenz Fraction:

(i) Efavirenz, microcrystalline cellulose, croscarmellose sodium, hydroxypropyl cellulose and low-substituted hydroxypropyl cellulose were sifted and blended together to form a dry blend,
(ii) Binder solution was prepared by dissolving sodium lauryl sulfate in purified water,
(iii) The blend of step (i) was granulated with solution of step (ii), using rapid mixer granulator,
(iv) The granules of step (iii) were dried and milled to get the desired granules of efavirenz.

B. Emtricitabine+Tenofovir Disoproxil Fumarate Fraction:

(i) Tenofovir disoproxil fumarate and microcrystalline cellulose were sifted and blended together to form a dry blend,
(ii) Binder solution was prepared by dissolving emtricitabine and hydroxypropyl methylcellulose in isopropyl alcohol,
(iii) The blend of step (i) was granulated with the with solution of step (ii), using fluid bed processor,
(iv) The granules of step (iii) were dried and milled to get the desired granules of emtricitabine and tenofovir disoproxil fumarate.

C. Extragranular Fraction and Compression:

(i) Efavirenz fraction of step A and emtricitabine and tenofovir disoproxil fumarate fraction of step B were blended together with extragranular croscarmellose sodium,
(ii) The blend of step (i) was lubricated with magnesium stearate,
(iii) The lubricated blend of step (ii) was compressed into single layer tablets using suitable compression machine.

D. Film Coating:

Single layer tablets obtained in Step C (iii) were film coated using the dispersion of Opadry II White 85F18422.

### Example-5

Tablet composition comprising efavirenz, emtricitabine and tenofovir disoproxil fumarate

| S. No | Ingredients | Qty (% w/w) |
|---|---|---|
| | A. Efavirenz Fraction: | |
| 1 | Efavirenz | 39.22 |
| 2 | Microcrystalline cellulose | 6.21 |
| 3 | Croscarmellose sodium | 2.94 |
| 4 | Hydroxypropyl cellulose | 1.31 |
| 5 | Low-substituted hydroxypropyl | 1.31 |
| | cellulose | |
| | Granulation: | |
| 6 | Sodium Lauryl Sulfate | 1.31 |
| 7 | Purified Water^{$} | q.s |
| | B. Emtricitabine Fraction: | |
| 8 | Emtricitabine | 13.07 |
| 9 | Microcrystalline cellulose | 2.61 |
| 10 | Croscarmellose sodium | 1.31 |
| | Granulation: | |
| 11 | Purified Water^{$} | q.s. |
| | C. Tenofovir disoproxil | |
| | fumarate Fraction: | |
| 12 | Tenofovir disoproxil fumarate | 19.61 |
| 13 | Microcrystalline cellulose | 3.92 |
| | Granulation: | |
| 14 | Hydroxypropyl methyl cellulose | 2.61 |
| | (Low viscosity grade) | |
| 15 | Isopropyl alcohol^{$} | q.s |
| 16 | Dichloromethane^{$} | q.s. |
| | D. Extragranular Fraction: | |
| 17 | Croscarmellose sodium | 3.59 |
| 18 | Magnesium Stearate | 0.98 |
| | Core tablet weight | 100.00 |
| | E. Film coating: | |
| 19 | Opadry II Pink 85F94172 | 3.00 |
| 20 | Purified water^{$} | q.s |

^{$}Lost in processing.

Manufacturing Process:

A. Efavirenz Fraction:

(i) Efavirenz, microcrystalline cellulose, croscarmellose sodium, hydroxypropyl cellulose and low-substituted hydroxypropyl cellulose were sifted and blended together to form a dry blend,
(ii) Binder solution was prepared by dissolving sodium lauryl sulfate in purified water,
(iii) The blend of step (i) was granulated with solution of step (ii), using rapid mixer granulator,
(iv) The granules of step (iii) were dried and milled to get the desired granules of efavirenz.

B. Emtricitabine Fraction:

(i) Emtricitabine, microcrystalline cellulose and croscarmellose sodium were sifted and blended together to form a dry blend,
(ii) The blend of step (i) was granulated with purified water, using rapid mixer granulator,
(iii) The granules of step (ii) were dried and milled to get the desired granules of emtricitabine.

C. Tenofovir Disoproxil Fumarate Fraction:

(i) Tenofovir disoproxil fumarate and microcrystalline cellulose were sifted and blended together to form a dry blend,
(ii) Hydroxypropyl methylcellulose solution was prepared in a mixture of isopropyl alcohol and dichloromethane,
(iii) The blend of step (i) was granulated with solution of step (ii), using fluid bed processor,
(iv) The granules of step (iii) were dried and milled to get the desired granules of tenofovir disoproxil fumarate.

D. Extragranular Fraction and Compression:

(i) Efavirenz fraction of step A, emtricitabine fraction of step B and tenofovir disoproxil fumarate fraction of step C were blended together with extragranular croscarmellose sodium,
(ii) The blend of step (i) was lubricated using magnesium stearate,
(iii) The lubricated blend of step (ii) was compressed using suitable compression machine.

E. Film Coating:

Single layer tablets obtained in Step D (iii) were film coated using the dispersion of Opadry II Pink 85F94172.

### Example-6 & 6a

Tablet composition comprising efavirenz, emtricitabine and tenofovir disoproxil fumarate

| | | Example 6 | Example 6a |
|---|---|---|---|
| S. No | Ingredients | Qty (% w/w) | Qty (% w/w) |
| | A. Efavirenz Fraction: | | |
| 1 | Efavirenz | 39.22 | 39.22 |
| 2 | Microcrystalline cellulose | 6.21 | 6.21 |
| 3 | Croscarmellose sodium | 2.94 | 2.94 |
| 4 | Hydroxypropyl cellulose | 1.31 | 1.31 |
| 5 | Low-substituted hydroxypropyl | 1.31 | 1.31 |
| | cellulose | | |
| | Granulation: | | |
| 6 | Sodium Lauryl Sulfate | 1.31 | 1.31 |
| 7 | Purified Water^{$} | q.s | q.s |
| | B. Emtricitabine Fraction: | | |
| 8 | Emtricitabine | 13.07 | 13.07 |
| 9 | Microcrystalline cellulose | 1.96 | 1.96 |
| 10 | Croscarmellose sodium | 1.96 | 1.96 |
| | Granulation: | | |
| 11 | Isopropyl alcohol^{$} | q.s. | q.s. |
| | C. Tenofovir disoproxil fumarate | | |
| | Fraction: | | |
| 12 | Tenofovir disoproxil fumarate | 19.61 | 19.61 |
| 13 | Microcrystalline cellulose | 4.57 | 4.57 |
| | Granulation: | | |
| 14 | Polyethylene glycol 8000 | 1.96 | 1.96 |
| 15 | Isopropyl alcohol^{$} | q.s | — |
| 16 | Purified water^{$} | — | q.s |
| | D. Extragranular Fraction: | | |
| 17 | Croscarmellose sodium | 3.59 | 3.59 |
| 18 | Magnesium Stearate | 0.98 | 0.98 |
| | Core tablet weight | 100.00 | 100.00 |
| | E. Film coating: | | |
| 19 | Opadry II Pink 85F94172 | 3.00 | 3.00 |
| 20 | Purified water^{$} | q.s | q.s |

^{$}Lost in processing.

Manufacturing Process:

A. Efavirenz Fraction:

(i) Efavirenz, microcrystalline cellulose, croscarmellose sodium, hydroxypropyl cellulose and low-substituted hydroxypropyl cellulose were sifted and blended together to form a dry blend,
(ii) Binder solution was prepared by dissolving sodium lauryl sulfate in purified water,
(iii) The blend of step (i) was granulated with solution of step (ii), using rapid mixer granulator,
(iv) The granules of step (iii) were dried and milled to get the desired granules of efavirenz.

B. Emtricitabine Fraction:

(i) Emtricitabine, microcrystalline cellulose and croscarmellose sodium were sifted and blended together to form a dry blend,
(ii) The blend of step (i) was granulated with isopropyl alcohol, using rapid mixer granulator,
(iii) The granules of step (ii) were dried and milled to get the desired granules of emtricitabine.

C. Tenofovir Disoproxil Fumarate Fraction:

(i) Tenofovir disoproxil fumarate and microcrystalline cellulose were sifted and blended together to form a dry blend,
(ii) Polyethylene glycol 8000 solution was prepared in isopropyl alcohol,
(iii) The blend of step (i) was granulated with solution of step (ii), using rapid mixer granulator,
(iv) The granules of step (iii) were dried and milled to get the desired granules of tenofovir disoproxil fumarate.

D. Extragranular Fraction and Compression:

(i) Efavirenz fraction of step A, emtricitabine fraction of step B and tenofovir disoproxil fumarate fraction of step C were blended together with extragranular croscarmellose sodium,
(ii) The blend of step (i) was lubricated using magnesium stearate,
(iii) The lubricated blend of step (ii) was compressed using suitable compression machine.

E. Film Coating:

Single layer tablets obtained in Step D (iii) were film coated using the dispersion of Opadry II Pink 85F94172.

### Example-6a

### Manufacturing Process Similar to Example-6

We claim:

1. Pharmaceutical composition comprising therapeutically effective amount of efavirenz, emtricitabine and tenofovir or pharmaceutically acceptable salt thereof as active ingredients from about 0.1% w/w to about 99% w/w based on total weight of the composition, optionally comprising one or more pharmaceutically acceptable excipient(s) from about 0.1% w/w to about 99% w/w based on total weight of the composition, wherein the said composition is in the form of a single layer tablet.

2. The composition according to claim 1, wherein the single layer tablet comprises about Tenofovir disoproxil fumarate, Efavirenz and Emtricitabine are each present in an amount from about 100 mg to about 600 mg.

3. The composition according to claim 1 or 2, wherein the single layer tablet comprises about 300 mg of Tenofovir disoproxil fumarate, about 600 mg of Efavirenz and about 200 mg of Emtricitabine.

4. The composition according to claims 1 to 3, wherein the said composition comprises a surfactant.

5. The composition according to claim 4, wherein the ratio of efavirenz to surfactant is about 50:1 to about 10:1.

6. The composition according to claims 1 to 5, wherein the tenofovir fraction is essentially free of surfactant(s).

7. The composition according to claim 1, wherein the pharmaceutically acceptable excipient(s) is selected from a group comprising diluents, binders, disintegrants, surfactants, glidant, lubricants, glidants/antiadherants; chelating agents; vehicles; bulking agents; stabilizers; preservatives used either alone or in combination thereof.

8. A process for the preparation of pharmaceutical compositions according to claim 1, wherein the process comprises of the following steps:
i) preparing the efavirenz fraction separately, optionally with one or more pharmaceutically acceptable excipient(s),
ii) preparing the emtricitabine fraction separately, optionally with one or more pharmaceutically acceptable excipient(s),
iii) preparing the tenofovir fraction separately, optionally with one or more pharmaceutically acceptable excipient(s), and
iv) formulating the material of steps (i), (ii) and (iii) into a single layer tablet.

9. A process for the preparation of pharmaceutical compositions according to claim 1, wherein the process comprises of the following steps:
i) preparing the efavirenz fraction separately, optionally with one or more pharmaceutically acceptable excipient(s),
ii) preparing the emtricitabine and tenofovir fractions together, optionally with one or more pharmaceutically acceptable excipient(s), and
iii) formulating the material of steps (i) and (ii) into a single layer tablet.

10. A process for the preparation of pharmaceutical compositions according to claim 8, wherein the process comprises of the following steps:
i) treating the efavirenz fraction separately with an aqueous or non-aqueous solvent, optionally with one or more other pharmaceutically acceptable excipient(s),
ii) treating the emtricitabine fraction separately with an aqueous or non-aqueous solvent, optionally with one or more other pharmaceutically acceptable excipient(s),
iii) treating the tenofovir fraction separately with an aqueous or non-aqueous solvent, optionally with one or more other pharmaceutically acceptable excipient(s), and
iv) formulating the material of steps (i), (ii) and (iii) into a single layer tablet.

11. A process for the preparation of pharmaceutical compositions according to claim 9, wherein the process comprises of the following steps:
i) treating the efavirenz fraction separately with an aqueous solvent or non-aqueous solvent, optionally with one or more other pharmaceutically acceptable excipient(s),
ii) treating the emtricitabine with an aqueous or non-aqueous solvent and tenofovir fractions together, optionally with one or more other pharmaceutically acceptable excipient(s), and
iii) formulating the material of steps (i) and (ii) into a single layer tablet.

12. A process for the preparation of pharmaceutical compositions according to claim 10, wherein the process comprises of the following steps:
i) treating the efavirenz fraction separately with an aqueous solvent, optionally with one or more other pharmaceutically acceptable excipient(s),
ii) treating the emtricitabine fraction separately with non-aqueous solvent, optionally with one or more other pharmaceutically acceptable excipient(s),
iii) treating the tenofovir fraction separately with non-aqueous solvent, optionally with one or more other pharmaceutically acceptable excipient(s), and
iv) formulating the material of steps (i), (ii) and (iii) into a single layer tablet.

13. A process for the preparation of pharmaceutical compositions according to claim 11, wherein the process comprises of the following steps:
i) treating the efavirenz fraction separately with an aqueous solvent, optionally with one or more other pharmaceutically acceptable excipient(s),
ii) treating the emtricitabine fraction with aqueous solvent and tenofovir fraction with a non-aqueous solvent together, optionally with one or more other pharmaceutically acceptable excipient(s), and
iii) formulating the material of steps (i) and (ii) into a single layer tablet.

14. A process for the preparation of pharmaceutical compositions according to claim 11, wherein the process comprises of the following steps:
i) treating the efavirenz fraction separately with an aqueous solvent, optionally with one or more pharmaceutically acceptable excipient(s),
ii) treating the emtricitabine with a non-aqueous solvent and tenofovir fractions together, optionally with one or more other pharmaceutically acceptable excipient(s), and
iii) formulating the material of steps (i) and (ii) into a single layer tablet.

15. Method for the prevention or treatment of patients infected with HIV according to claim 1 that provides enhanced therapeutic safety and efficacy, impart lower resistance, and results in higher patient compliance.
